(19) United States
(12) Patent Application Publication
(10) Pub. No.: US 2022/0238226 A1
(43) Pub. Date: Jul. 28, 2022

(54) METHOD FOR PREDICTING MEDICINAL EFFECTS OF COMPOUNDS USING DEEP LEARNING

(71) Applicants: INDUSTRY FOUNDATION OF CHONNAM NATIONAL UNIVERSITY, Gwangju (KR); BIO-SYNERGY RESEARCH CENTER, Daejeon (KR); KOREA ADVANCED INSTITUTE OF SCIENCE AND TECHNOLOGY, Daejeon (KR)

(72) Inventors: Sunyong YOO, Gwangju (KR); Doheon LEE, Daejeon (KR)

(21) Appl. No.: 17/564,874

(22) Filed: Dec. 29, 2021

(30) Foreign Application Priority Data
Jan. 28, 2021 (KR) 10-2021-0012339

(51) Int. Cl. G16H 50/20; G06N 3/08; G06N 3/04

(52) U.S. Cl. CPC G16H 50/20; G06N 3/04; G06N 3/08

(57) ABSTRACT

Disclosed is a method for predicting medicinal effects wherein medicinal effects of novel compounds are predicted by generating three types of feature data from acquired medicinal substance data, training a neural network model, and then applying acquired new compound data to the neural network model, and the use of the present disclosure mitigates the bottleneck effect of deep learning models and thus the present disclosure can be used to perform a large-scale natural compound study and can perform a preliminary screening of compounds for a large number of candidate medicinal substances, with a high accuracy of medicinal effect prediction.

### CROSS-REFERENCE TO RELATED APPLICATIONS

This application claims the benefit and priority to Korean Patent Application No. 10-2021-0012339, filed on Jan. 28, 2021. The entire disclosure of the application identified in this paragraph is incorporated herein by references.

### FIELD

The present invention relates to a method for predicting medicinal effects, wherein medicinal effects of novel compounds are predicted by generating three types of feature data from acquired medicinal substance data, training a neural network model using the feature data, and then applying acquired new compound data to the neural network model.

### BACKGROUND

Medicinal plants possess diverse natural compounds, contributing to drug development by providing novel candidate therapeutic agents against various diseases. Natural compounds are small molecules synthesized by living organisms, including primary and secondary metabolites. The ingestion of bioactive natural compounds, such as phytochemicals, antioxidants, vitamins, and minerals, may promote health via negative immunoregulatory and anti-inflammatory activities. Many natural compounds have been proven to play an important role as modulators of cell signaling and homeostasis, which enforces the need to identify the medicinal potentials of bioactive natural compounds.

In most of the previous studies, in vitro screening tests were performed for the assessment of the biological activities of natural compounds. However, largescale experiments are needed as the number of considered natural compounds and candidate effects increases, which exponentially increases experimental time and cost. Therefore, in silico approaches, which mostly focus on specific information such as molecular properties, chemical similarities, or clinical knowledge, have been proposed to predict medicinal candidates from natural compounds.

Molecular-based approaches focus on finding similar responses or mechanisms between natural compounds and drugs from various networks, e.g., functional protein interactions or compound-target interactions. Chemical-based approaches investigate bioactive natural compound candidates by examining physicochemical properties and physiological effects. However, the molecular targets, mechanisms, and chemical structure information of natural compounds are largely hidden, compared with those of approved drugs. Therefore, both molecular and chemical-based approaches have low coverage and usability.

Knowledge-based approaches apply statistical analysis to scientific databases, such as PubMed, or clinical investigational information to identify medicinal natural compound candidates for a certain disease. These approaches provide better coverage compared with molecular and chemical-based approaches, but their performance is low because they cannot directly consider complex molecular mechanisms and chemical structures.

Alternatively, machine learning-based approaches were proposed to utilize large volume of information. These approaches predicted the potential effects of natural compounds by investigating the drugs having similar properties to those of natural compounds and applying the investigation results to a prediction model employing classification algorithms.

However, limited natural compound information is still a bottleneck effect when trying to utilize various types of features in the learning process of learning natural compound information in prediction models. Therefore, there is a need for a new approach that can solve the bottleneck effect while utilizing the limited information of natural compounds.

### SUMMARY

The present inventors have endeavored to make a deep learning model capable of precisely predicting medicinal effects of natural compounds even when the limited, heterogeneous, and incomplete information on the natural compounds is used.

As a result, the present inventors identified that the medicinal effects of natural compounds could be predicted with high accuracy by using the natural compound and approved investigational drug information to learn a deep learning model to which a partially connected deep neural network is applied.

Accordingly, an aspect of the present invention is to provide a method for predicting medicinal effects of compounds by using deep learning.

Another aspect of the present invention is to provide a computer program for predicting medicinal effects of compounds by using deep learning.

Still another aspect of the present invention is to provide a system for predicting medicinal effects of compounds by using deep learning.

The present invention relates to a method for predicting medicinal effects, wherein medicinal effects of novel compounds are predicted by generating three types of feature data from acquired medicinal substance data, training a neural network model using the feature data, and then applying acquired new compound data to the neural network model.

Hereinafter, the present disclosure will be described in more detail.

In accordance with an aspect of the present disclosure, there is provided a method for predicting medicinal effects of compounds by using deep learning, the method including:

a data acquirement step of acquiring medicinal substance data;

a feature generation step of generating feature data from the acquired medicinal substance data;

a training step of training a neural network model including an input layer, hidden layers, and an output layer as feature data; and

a prediction step of predicting medicinal effects of compounds by applying compound data to the neural network model.

In the present invention, the medicinal substance data may be acquired from at least one database selected from the group consisting of DrugBank, Common Technical Document (CTD), Manually Annotated Targets and Drugs Online Resource (MATADOR), STITCH, and Therapeutic Target Database (TTD), but is not limited thereto.

In the present invention, the medicinal substance data may include information of names, chemical structures, and medicinal effects of medicinal substances, but are not limited thereto.

In the present invention, the feature data may mean features that the neural network model needs to notice from given data.

The neural network model may predict medicinal effect candidates of compounds by using the feature data.

In the present disclosure, the feature data may have a fixed-length numeric vector form.

In the present disclosure, the feature data may include latent knowledge features, molecular interaction features, and chemical property features.

In the present invention, the latent knowledge features may be generated by extraction from scientific literature and the like through word embedding.

The word embedding is one of the language models, and may be characterized by analyzing the relationship between words within a sentence in an unsupervised learning manner.

In the present disclosure, the word embedding may be performed using at least one selected from the group consisting of Word2vec, AdaGram, fastText, and Doc2vec, but is not limited thereto.

The fastText may use the sub-word skip-gram model that learns representations for character n-grams based on unlabeled corpora where each word is represented as the sum of the n-gram vector representations.

In an embodiment of the present invention, the latent knowledge features may be generated by extraction of words from the National Library of Medicine National Center for Biotechnology Information (NCBI) PubMed abstract.

In the present disclosure, the molecular interaction features may be generated by constructing a protein-protein interaction (PPI) network from the acquired compound data and medicinal substance data and applying a random walk with restart (RWR) algorithm thereto.

The RWR algorithm may simulate the random walker starting from seed nodes of the deep learning model and iteratively diffuse the node values to the neighbors according to edge weights until stability is achieved.

In the present invention, the molecular interaction features may be generated using at least one type of information selected from the group consisting of direct binding information and indirect binding information for the proteins of the compounds, and may be generated using, for example, direct binding and indirect binding information, but is not limited thereto.

The direct binding may indicate the target proteins of the compounds.

The indirect binding may indicate the molecular effects of the compounds, including changes in protein expression and compound-induced phosphorylation, or the effects of compounds that are transformed into active metabolites.

In the present invention, chemical property features may be generated through SwissADME.

In the present invention, the chemical property features may include physicochemical property, lipophilicity, solubility, pharmacokinetics, drug-likeness, and medicinal chemistry friendless information.

In the present invention, the physicochemical property information may contain molecular weight, number of heavy atoms, fraction Csp3, rotatable bonds, hydrogen-bond acceptors, hydrogen-bond donors, and molar refractivity.

In the present invention, the lipophilicity information may contain the results of five methods (XLOGP3, WLOGP, MLOGP, SILICOS-IT, and iLOGP) for the prediction of the partition coefficient between n-octanol and water (log Po/w).

In the present invention, the solubility information may contain the results of three different methods for the prediction of solubility, such as estimated solubility (ESOL), Ali, and SILICOS-IT.

In the present invention, the pharmacokinetics information may contain human intestinal absorption, blood-brain barrier permeability, permeability glycoprotein (P-gp) substrate, five major isoforms of cytochrome P450 (i.e., CYP1A2, CYP2C19, CYP2C9, CYP2D6, and CYP3A4), and the logarithm of skin permeability coefficient (log Kp).

In the present invention, the drug-likeness information may contain Lipinski's rule of five, Ghose, Veber, Egan, Muegge, and bioavailability score.

In the present invention, the medicinal chemistry friendless information may contain the pan assay interference compounds (PAINS) filter, the Brenk filter, lead-likeness, and synthetic accessibility values.

In the present invention, the neural network model may include an input layer, hidden layers, and an output layer.

In an embodiment of the present invention, the input layer, hidden layers, and output layer may be arranged in that order in the neural network model.

In an embodiment of the present invention, the hidden layers may include partially connected layers and fully connected layers.

The partially connected layer may mean a layer including only a subset of each connectable set of the neural network model. The partially connected layer may reduce complexity and improve generalization without producing modeling errors.

The fully connected layer may mean a layer that constitutes a complete connection between layers at the latter part of the layer. The fully connected layer may simplify the model design since every neuron in one layer is connected to every neuron in the next layer, but may need large training data and may not consider the characteristic of the input feature types.

In an embodiment of the present invention, the input layer, partially connected layers, fully connected layers, and output layer may be arranged in that order in the neural network model.

In the present disclosure, the hidden layers may include rectified linear unit (ReLU) and batch normalization functions, but are not limited thereto.

In the present invention, the rectified linear unit function may be applied to the hidden units of the neural network model to increase the nonlinearity. The weight of the neural network model may be initialized using random numbers considering the increased ReLU nonlinearity.

In the present invention, the batch normalization may be used to normalize the input layer.

In the present invention, the training step may be training a neural network model including an input layer, hidden layers, and an output layer as feature data, but is not limited thereto.

In the present invention, the training step may be inputting the feature data into the input layer and learning medicinal effect information matching the feature data through the hidden layers, but is not limited thereto.

In the present invention, the prediction step may be inputting compound data into the input layer and allowing the neural network model to predict medicinal effect candidates of the compounds.

In the present disclosure, the compound may be at least one selected from the group consisting of natural compounds and synthetic compounds, but is not limited thereto.

In the present disclosure, the compound data may be acquired from at least one type of database selected from the group consisting of Korean Traditional Knowledge Portal (KTKP), Traditional Chinese Medicine Integrated Database (TCMID), Compound Combination-Oriented Natural Product Database with Unified Terminology (COCONUT), and Food Database (FooDB).

In an embodiment of the present disclosure, when new compound data are input to the input layer, the neural network model may calculate drug effect information that matches the new compound data through the output layer learning the medical effect information, thereby calculating drug effect data.

The present disclosure has a wide coverage of predictable compounds by utilizing various information corresponding to latent knowledge, intermolecular interactions, and chemical properties. Therefore, the use of the present disclosure can mitigate the bottleneck effect of most of the existing in silico models that utilize only specific information and cannot predict the result without the information to use, and also can improve prediction performance.

Another aspect of the present disclosure relates to a computer program, recorded on a computer-readable recording medium, to implement a method for predicting medicinal effects of compounds in conjunction with a computer system.

In an embodiment of the present disclosure, the computer program may independently or collectively instruct or configure the processing device to operate as desired.

In an embodiment of the present disclosure, the computer program may be embodied permanently or temporarily in any type of machine, component, physical or virtual equipment, or computer storage medium or device, in order to provide instructions or data to or be interpreted by the processing device. Especially, the software also may be distributed, stored, or implemented over network coupled computer systems. Such a computer program may be stored by one or more computer-readable recording media.

In an embodiment of the present disclosure, the prediction method of the disclosure may be implemented in a type of program instruction that can be performed through various computer implementation means, and may be recorded on a computer-readable medium. The medium may continuously store the computer-executable programs or instructions, or temporarily store the computer-executable programs or instructions for execution or downloading. Also, the medium may be any one of various recording media or storage media in which a single piece or plurality of pieces of hardware are combined, and the medium is not limited to a medium directly connected to a computer system, but may be distributed on a network.

Examples of the medium include magnetic media, such as a hard disk, a floppy disk, and a magnetic tape, optical recording media, such as CD-ROM and DVD, magneto-optical media such as a floptical disk, and ROM, RAM, and a flash memory, which are configured to store program instructions, but are not limited thereto.

Other examples of the medium include recording media and storage media managed by application stores distributing applications or by websites, servers, and the like supplying or distributing other various types of software, but are not limited thereto.

The program instructions recorded on media may be specially designed and configured for the purposes of the exemplary embodiments, or may be well-known and available to a person skilled in the art. Examples of program code include machine codes produced by a compiler as well as higher-level program codes that can be executed by a computer using an interpreter or the like.

In accordance with still another aspect, there is provided a computer-implemented system for predicting medicinal effects of compounds,

the computer including at least one processor configured to execute computer-readable instructions, wherein the at least one processor:

acquires medicinal substance data;

generates feature data from the acquired medicinal substance data;

trains a neural network model including an input layer, hidden layers, and an output layer as feature data; and

predicts medicinal effects of compounds by applying compound data to the neural network model.

The system of the present disclosure may include a program or processor to perform the above-described method for predicting medicinal effects.

The present disclosure relates to a method for predicting medicinal effects of compounds by using deep learning, and the use of the present disclosure mitigates the bottleneck effect of the existing in silico models by utilizing large amounts of heterogeneous information containing latent knowledge, molecular interactions, and chemical properties, to mitigate the incomplete information, and thus, the present disclosure can be used to perform a large-scale natural compound study.

Furthermore, the present disclosure can perform a preliminary screening of compounds for a large number of candidate medicinal substances, with a high accuracy of medicinal effect prediction.

### BRIEF DESCRIPTION OF THE DRAWINGS

FIG. 1 shows three features that a deep learning model uses to predict the medicinal effects of natural compounds according to an embodiment of the present disclosure.

FIGS. 2A, 2B, 2C, 2D, 2E, 2F, 2G, 2H, 2I, 2J, 2K, 2L, 2M, 2N, 2O, 2P, 2Q and 2R shows the comparison results of the distribution of chemical properties between natural compounds and drugs.

FIG. 3 schematically illustrates the schematic structure of the deep learning model of the present disclosure.

FIG. 4 shows feature data used by the deep learning model of the present disclosure.

FIG. 5 shows a graph comparing the AUROC value for 15 diseases, predicted by the deep learning model according to an embodiment of the present disclosure with the AUROC values by the neural network models composed of fully connected neural networks and having different feature combinations.

FIG. 6 is a graph comparing the AUROC value for 15 diseases, predicted by the deep learning model according to an embodiment of the present disclosure with the AUROC values by other machine learning methods, such as logistic regression, support vector machine (SVM), and bootstrapping.

### DETAILED DESCRIPTION

Hereinafter, the present disclosure will be described in more detail with reference to exemplary embodiments. These exemplary embodiments are provided only for the purpose of illustrating the present disclosure in more detail, and therefore, according to the purpose of the present disclosure, it would be apparent to a person skilled in the art that these exemplary embodiments are not construed to limit the scope of the present disclosure.

In some cases, known structures and devices may be omitted or block diagrams mainly illustrating key functions of the structures and devices may be provided so as not to obscure the concept of the present disclosure. Throughout the specification, like reference numerals will be used to refer to like elements.

Throughout the specification, when a part is referred to as “comprising” or “including” an element, this indicates that the part may further include another element instead of excluding another element unless particularly stated otherwise.

The term “ . . . unit” used herein refers to a unit that performs at least one function or operation and may be implemented in hardware, software, or a combination thereof. Furthermore, “a” or “an”, “one”, and the like may be used to include both the singular form and the plural form unless indicated otherwise in the context of the present disclosure or clearly denied in the context.

Hereinafter, preferable embodiments of the present disclosure will be described with reference to the accompanying drawings. A detailed description to be disclosed below together with the accompanying drawings is to describe the exemplary embodiments of the present disclosure and does not represent the sole embodiment for carrying out the present disclosure.

### Experimental Example 1: Data Collection

Plant-derived natural compounds and their chemical structure information were collected from Korea Traditional Knowledge Portal (KTKP), Traditional Chinese Medicine Integrated Database (TCMID), Compound Combination-Oriented Natural Product Database with Unified Terminology (COCONUT), and the Food Database (FooDB).

Drug information, containing chemical structure and indication, was collected from DrugBank version 5.1.5. The molecular targets of the drugs and natural compounds were collected from the DrugBank, Common Technical Document (CTD), Manually Annotated Targets and Drugs Online Resource (MATADOR), STITCH, and Therapeutic Target Database (TTD).

In the present disclosure, 4,507 natural compounds and 2,882 approved and investigational drugs that have at least five molecular target information. For extracting latent knowledge from scientific literature and the like, 13,200,786 PubMed abstracts that were published from 1950 to 2019, containing 236,645,741 sentences and 3,689,111,651 words were collected.

For the molecular interaction analysis, a protein-protein interaction (PPI) dataset was obtained from BioGrid version 3.5.182, containing 18,008 nodes and 504,848 edges.

### Experimental Example 2: Generating Heterogeneous Features of Drugs and Natural Compounds

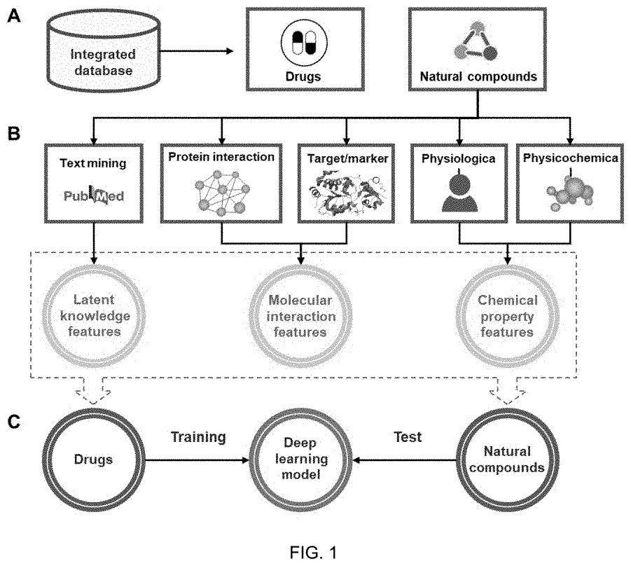

To predict the medicinal effects of natural compounds, three important features were generated (FIG. 1). Each feature was generated by a fixed-length numeric vector form.

2-1. Latent Knowledge Features

Latent knowledge features need to be generated to obtain various types of natural compound and drug information from scientific literature. For the generation of the latent knowledge features, a word embedding approach that represents a single word as a real-valued vector in a low-dimensional space was applied (FIG. 1A).

For text mining, the fastText was used. The fastText improves the representations of rare words by considering the character level information and the internal structure of the words. For example, the natural compound name “alpha-isothiocyanatotoluene” can be estimated by dividing the word into “alpha”, “isothiocyanato”, and “toluene,” which are relatively frequent in the training corpora. The fastText model learns the distributed representations for all character n-grams in “alphaisothiocyanatotoluene” and integrates the sub-word vectors to generate the final embedding vector of “alphaisothiocyanatotoluene”.

The deep learning model of the present disclosure used the pre-trained fastText model with Wikipedia and Common Crawl. The model additionally learned the DrugBank medicinal effect information and PubMed literature. Before training, all the words and sentences included in each literature were tokenized and transformed into lowercase, and then special characters and Greek symbols were transformed into alphabetic names (e.g., a to alpha).

2-2. Molecular Interaction Features

Molecular interaction features were generated by investigating mechanisms from the binding targets of compounds to the therapeutic targets or biomarkers of diseases. To this end, a protein-protein interaction (PPI) network was constructed and the random walk with restart (RWR) algorithm was applied to quantify the molecular interaction effects of the compounds (FIG. 1B).

The RWR simulates the random walker starting from seed nodes and iteratively diffuses the node values to the neighbors according to edge weights until stability is achieved. The RWR is defined as equation 1.

pₜ₊₁=(1−r)W^{T}pₜ+rp₀ [Equation 1]

where W is the column-wise normalized adjacency matrix of the network, and r is the restarting probability of the random walker at each time step (it was set to 0.7 in the present disclosure). The adscript of pₜ represents the probability vector of each node at time step t, and p0 represents the initial probability vector. To apply the RWR algorithm, the initial values of the seed nodes were first set based on the binding target information of the compounds.

The deep learning model of the present disclosure used two types of binding target information: direct binding and indirect binding. The direct binding indicates the target proteins of the compounds, whereas the indirect binding indicates the molecular effects of the compounds, including changes in protein expression and compound-induced phosphorylation, or the effects of compounds that are transformed into active metabolites. By considering both types of binding information, various properties of the compounds on the network can be considered. The initial values (p₀) of the direct binding and indirect binding were assigned as 1 and 0.3, respectively.

Next, the transition probability from a node to the neighbors was calculated. It was assumed that the transition probability represents the propagated effects on the PPI network. Based on equation 1, the transition probability vector of each node at time step t+1 was calculated. The RWR algorithm simulated the random walker until pₜ became stable, which was evaluated by ∥pₜ₊₁−pₜ∥<10⁻⁸. In the present disclosure, 4,487 disease-related proteins were considered from a total of 18,008 proteins that were collected.

However, principal component analysis (PCA) was performed on the probability vector of proteins to reduce the dimensionality (i.e., from 4,487 to 285), as the number of proteins was still large compared with the number of instances of the training set. The threshold of the cumulative explained variance ratio was set as 0.8. Based on the PCA results, molecular interaction features were generated.

2-3. Chemical Property Features

Chemical property features were generated by considering physicochemical property, lipophilicity, solubility, pharmacokinetics, drug-likeness, and medicinal chemistry friendless information (FIG. 1C).

Physicochemical properties include molecular weight, number of heavy atoms, fraction Csp3, rotatable bonds, hydrogen-bond acceptors, hydrogen-bond donors, and molar refractivity. For all physicochemical properties, feature scaling was performed by applying Z-score normalization. The scale of input variables used to train the model is an important factor because unscaled inputs can result in a slow or unstable learning process to cause exploding gradients in the learning process. Therefore, Z-score normalization was performed that can standardize the values having a mean of 0 and a standard deviation of 1, unit variance.

Lipophilicity contains the results of five different methods (XLOGP3, WLOGP, MLOGP, SILICOS-IT, and iLOGP) for the prediction of the partition coefficient between n-octanol and water (log Po/w). The consensus log Po/w is the arithmetic mean of the values predicted by the above five methods.

Solubility includes the results of three different methods for the prediction of solubility, containing the ESOL, Ali, and SILICOS-IT methods.

Pharmacokinetics includes human intestinal absorption, blood-brain barrier permeability, permeability glycoprotein (P-gp) substrate, five major isoforms of cytochrome P450 (i.e., CYP1A2, CYP2C19, CYP2C9, CYP2D6, and CYP3A4), and the logarithm of skin permeability coefficient (log Kp). Drug-likeness contains Lipinski's rule of five, Ghose, Veber, Egan, Muegge, and bioavailability score.

The lipophilicity, solubility, pharmacokinetics, and drug-likeness values were used without feature scaling because the data are log scale or the data type was categorical. All categorical data were transformed into binary variables by applying one-hot encoding.

Lastly, medicinal chemistry friendless contains the pan assay interference compounds (PAINS) filter, the Brenk filter, lead-likeness, and synthetic accessibility.

All the properties were calculated using SwissADME.

### Experimental Example 3: Evaluation of Features

3-1. Latent Knowledge Features

The latent knowledge features were evaluated by calculating the similarity for groups of drugs based on the Anatomical Therapeutic Chemical (ATC) code. The ATC classification system categorizes drugs into different groups according to their chemical, pharmacological, and therapeutic properties. In the ATC classification system, drugs are classified into groups at five different levels: the first level has 14 anatomical main groups; the second level indicates the main therapeutic group; the third level indicates a therapeutic or pharmacological subgroup; the fourth level indicates a therapeutic, pharmacological, or chemical subgroup; and the fifth level is the chemical substance.

The drugs were grouped based on the five levels of the ATC code. For each group, cosine similarity values for the latent knowledge features of all possible drug pairs were calculated, and as a result, it was found that the mean value of the cosine similarity of the same ATC code group (S₁ₛₜ=0.417, S_{2nd}=0.478, S_{3rd}=0.551, S₄ₜₕ=0.603, and S₅ₜₕ=0.608) was higher than that of the randomly selected group (S_{random}=0.341-0.369). Moreover, it was confirmed that the similarity of the latent knowledge features increased as the level of ATC codes was higher.

Such cosine similarity was higher than the similarity value calculated by word2vec. (Cosine similarity of word2vec: S₁ₛₜ=0.322, S_{2nd}=0.349, S_{3rd}=0.423, S₄ₜₕ=0.498, and S₅ₜₕ=0.502). These results indicated that the latent knowledge features effectively represented the anatomical, therapeutic, and pharmacological properties, as the deeper the ATC level, the more similar the properties of the drugs.

3-2. Molecular Interaction Features

It was confirmed whether the molecular interaction features can be used to predict the potential medicinal effects of compounds. To this end, the sum of the protein values of the molecular interaction features was mapped to diseases based on the therapeutic target and biomarker information of diseases. Target diseases include 3,832 diseases defined by MeSH and Online Mendelian Inheritance in Man (OMIM). Through this process, a list of disease scores for each drug was obtained.

The prediction results were compared with the results of the network-based efficacy screening methods, including closest, shortest, kernel, center, and separation methods. The closest method predicts effects by calculating the mean shortest distance between compound targets and the nearest disease gene. The shortest method calculates the mean shortest distance between all compound targets and disease-related proteins. The kernel method calculates the distance by down-weighting long paths exponentially. The center method calculates distance with considering the largest closeness centrality among the disease-related proteins. Lastly, the separation method calculates the sum of the mean distance between compound targets and disease-related proteins using the closest method and subtracts the sum from the mean shortest distance between compound targets and disease-related proteins.

As for the medicinal effects prediction using the molecular interaction features, the area under the ROC curve (AUROC) was measured to be 0.776±0.094, and thus exhibited better performance compared with the medicinal effects prediction using the closest (AUROC=0.721±0.076), shortest (AUROC=0.697±0.102), kernel (AUROC=0.713±0.084), center (AUROC=0.707±0.088), and separation (AUROC=0.710±0.078) methods. These results indicates the effectiveness of the molecular interaction features in predicting the effects of compounds by analyzing propagated effects compared with the conventional approach.

3-3. Chemical Property Features

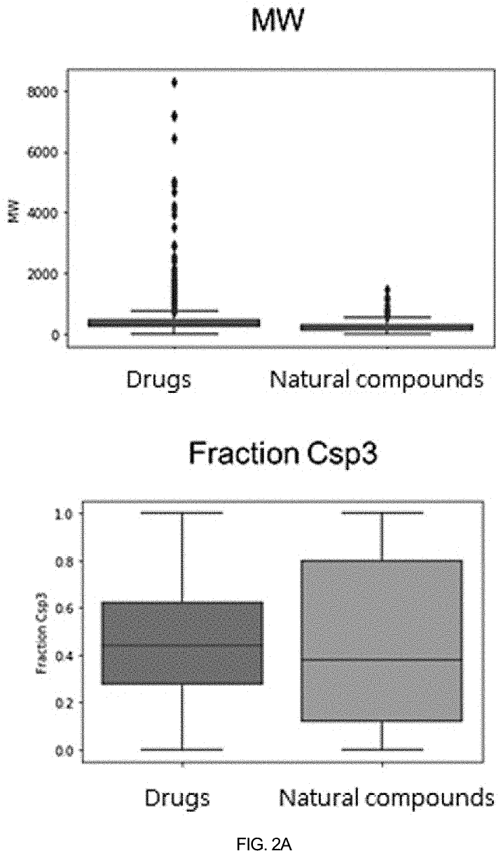

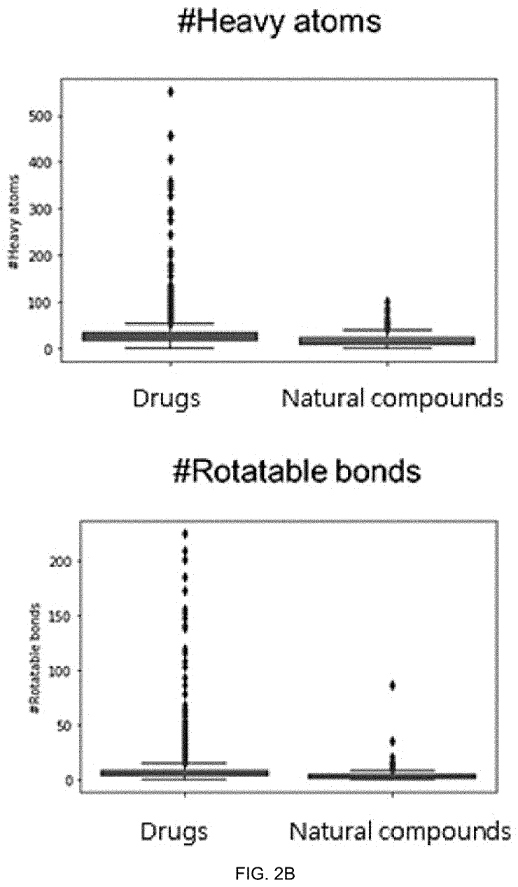

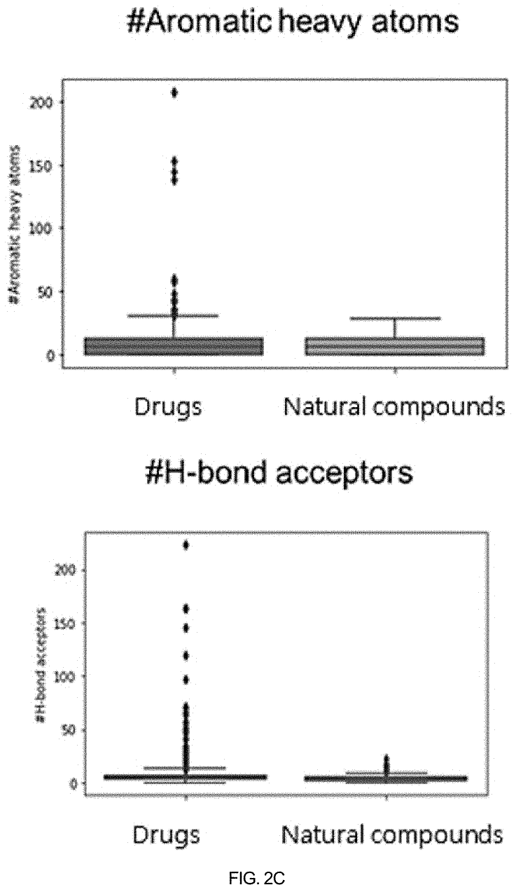

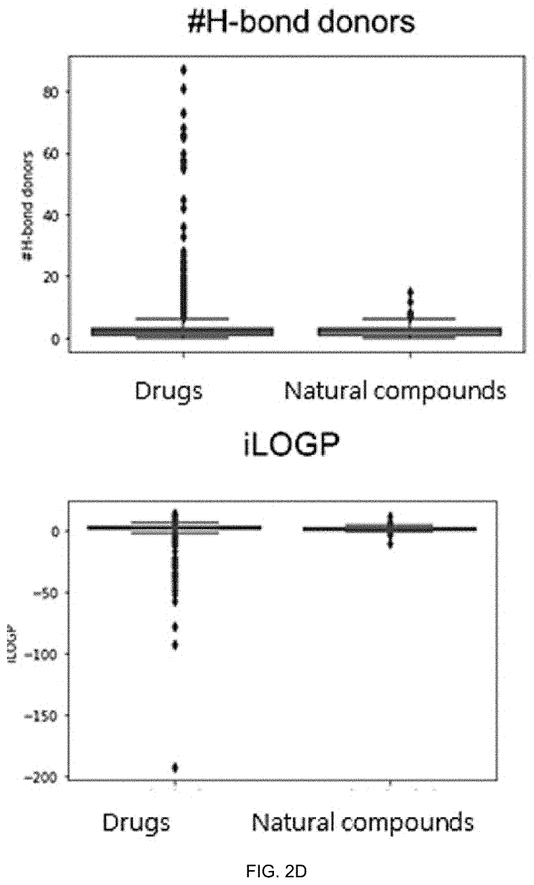

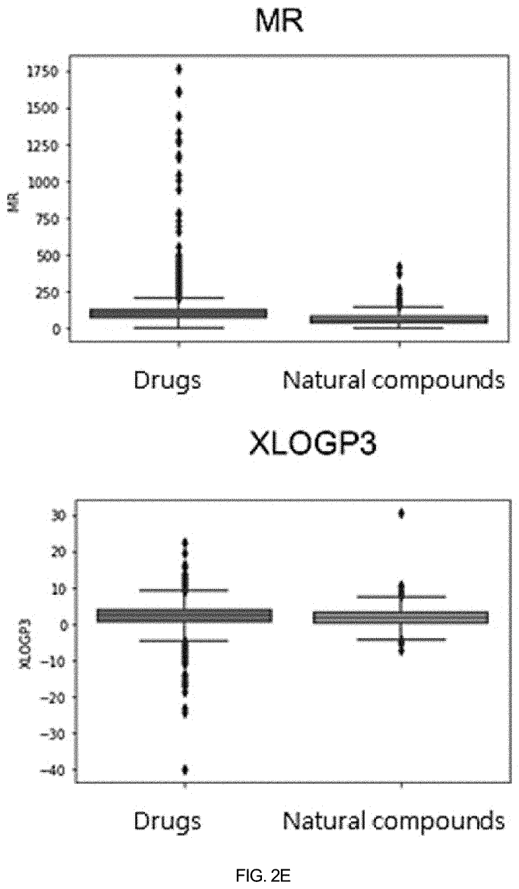

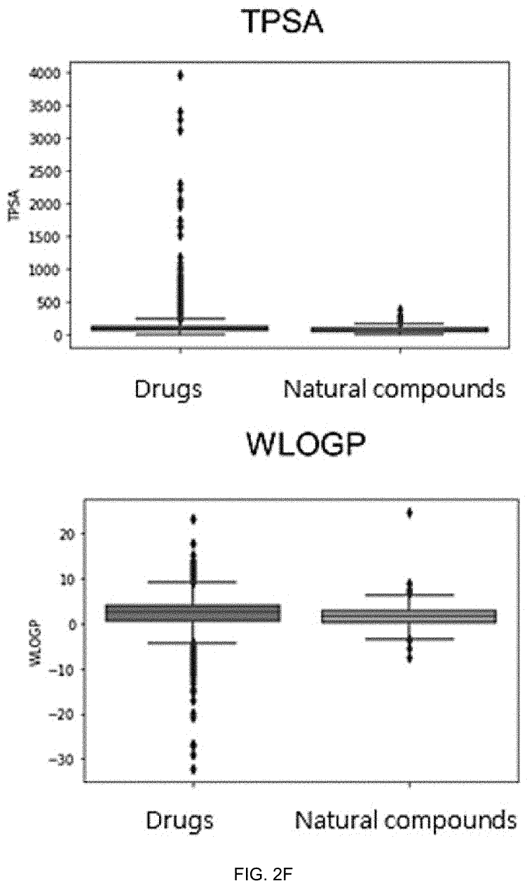

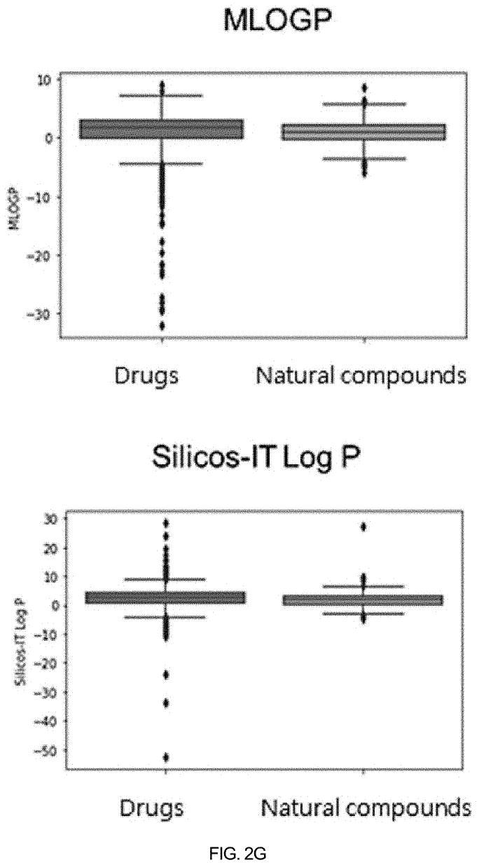

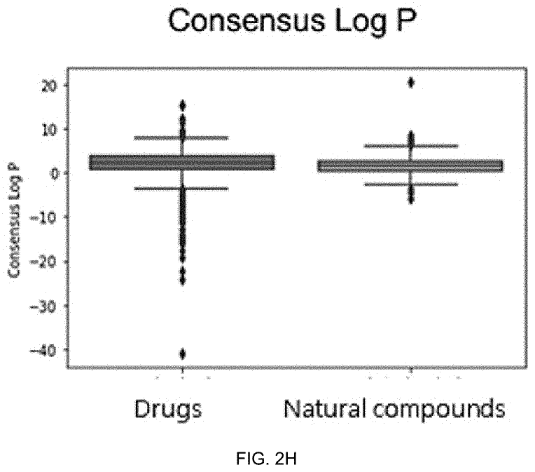

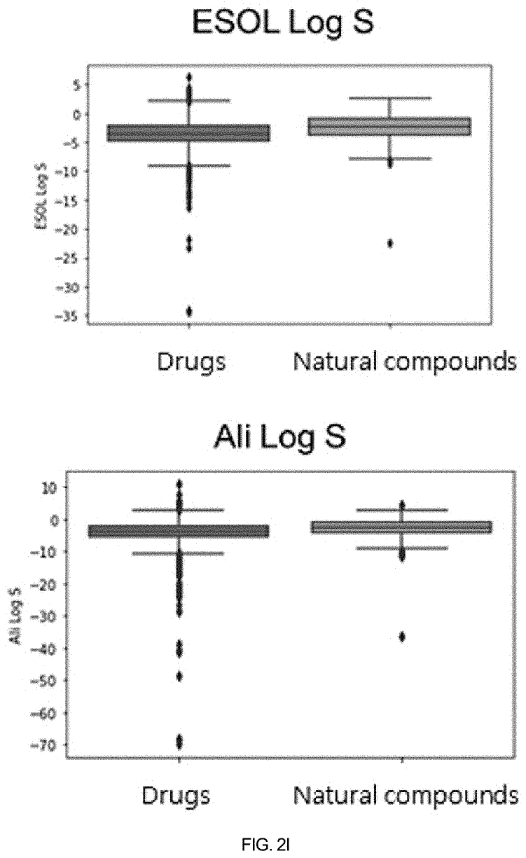

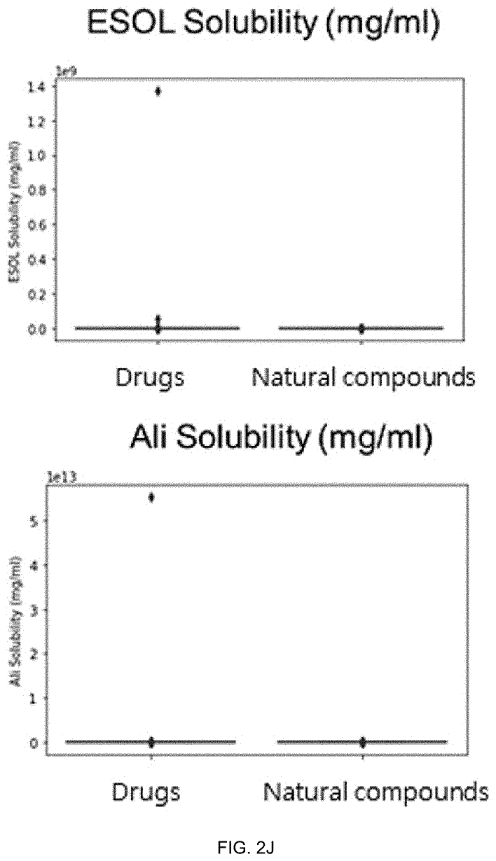

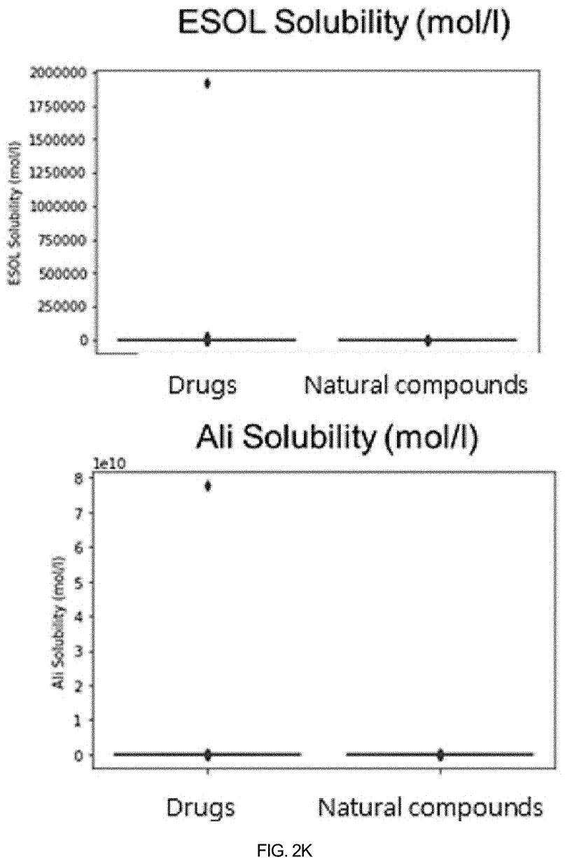

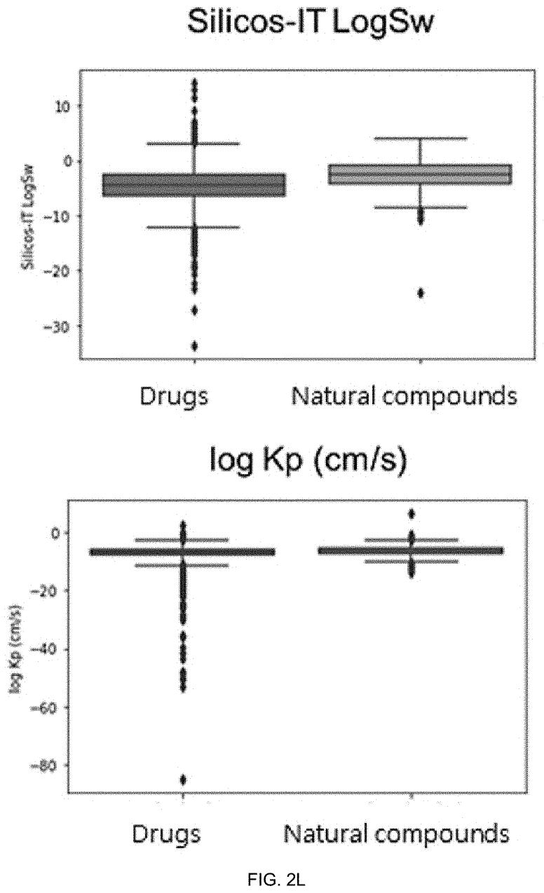

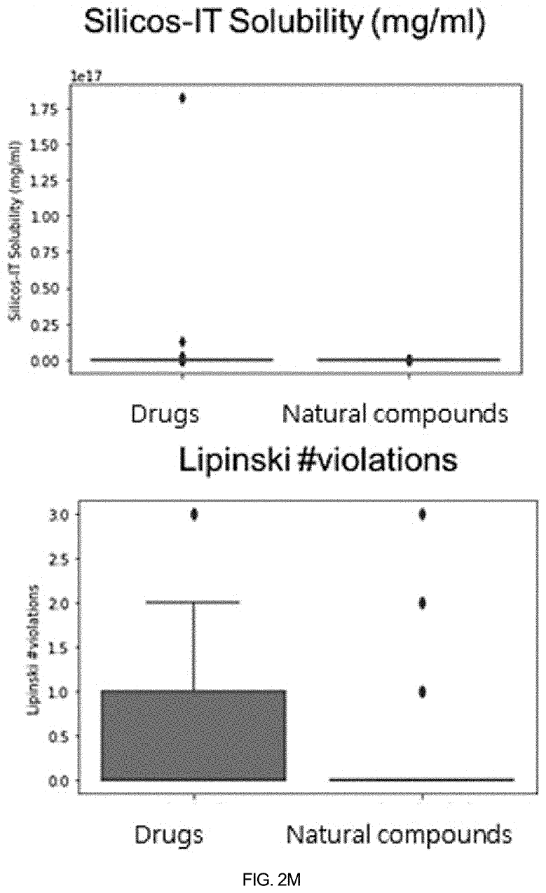

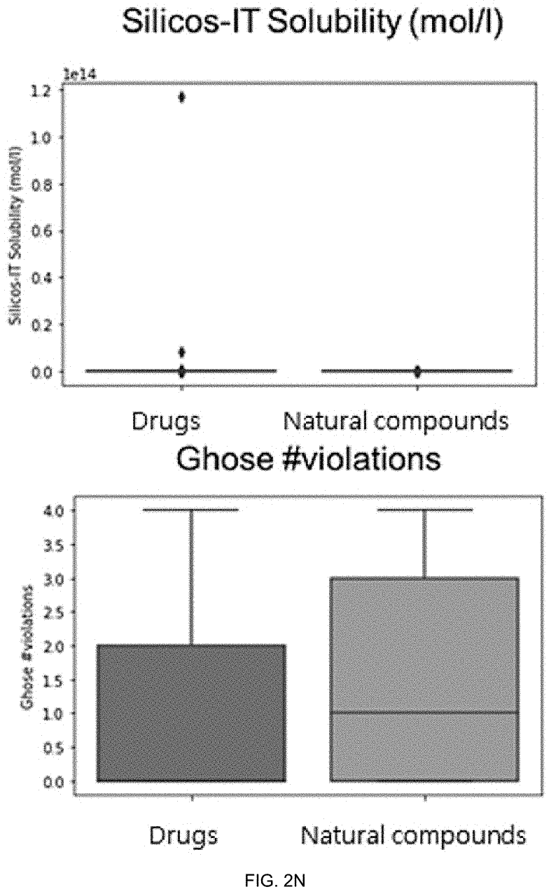

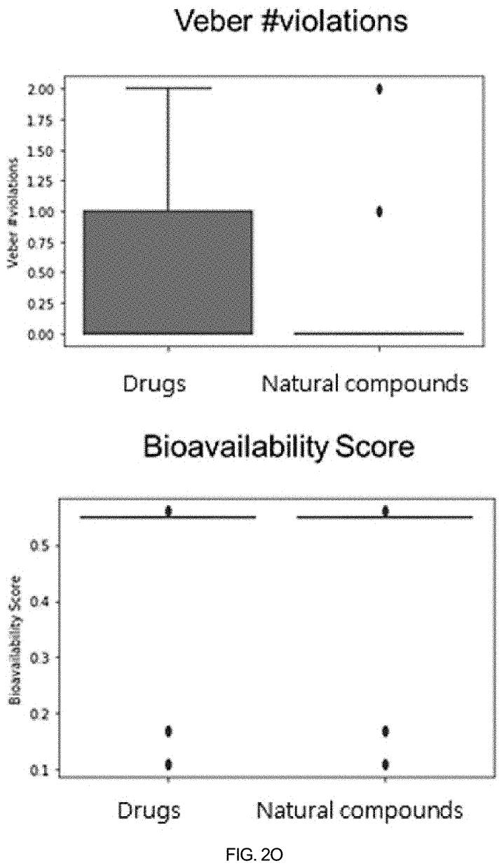

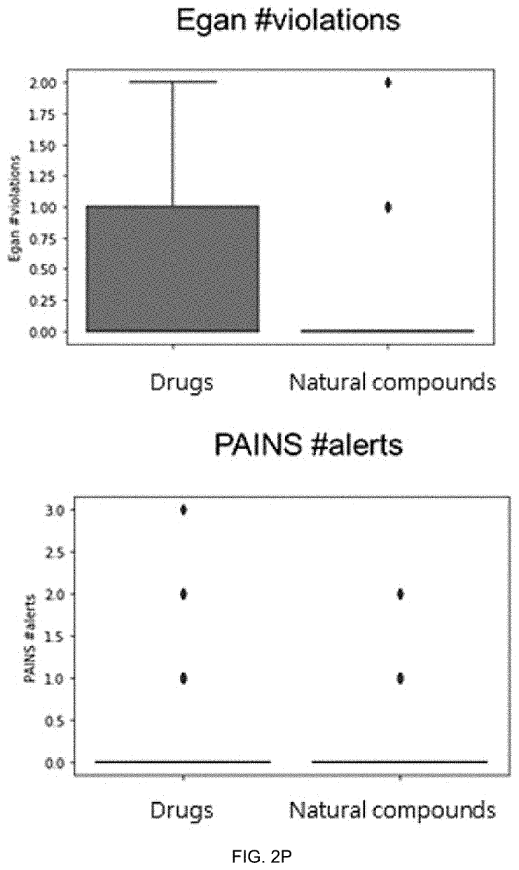

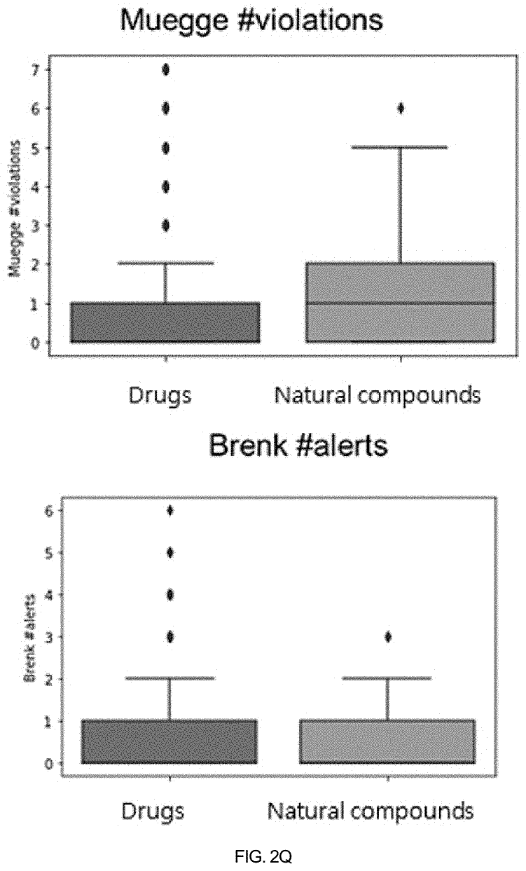

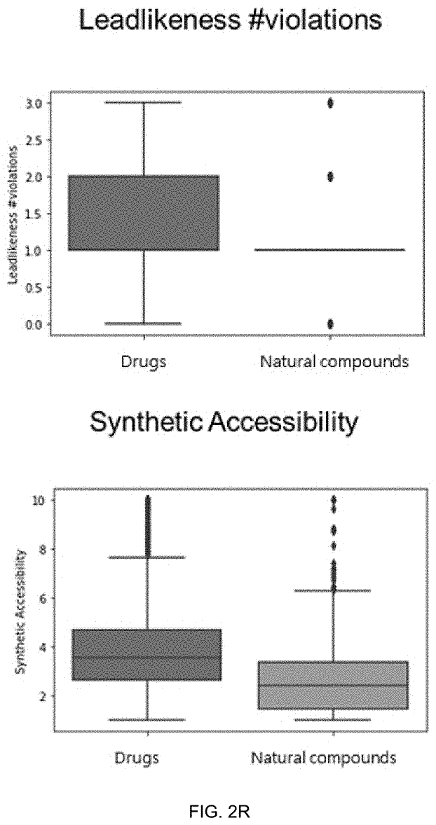

Various statistical tests were performed to analyze the chemical property features. Firstly, the comparison results of the distribution of the chemical properties of the natural compounds and drugs are shown in FIGS. 2A to 2R.

As confirmed in FIGS. 2A to 2R, the median values of 68% chemical properties of natural compounds lie inside of the interquartile range of drugs. The mean, standard deviation, and standard error of the chemical properties of the natural compounds and drugs are provided in Table 1.

TABLE 1

| | Natural compounds | | | DrugBank | | |
|---|---|---|---|---|---|---|
| | | | Standard | | | Standard |
| | | Standard | error of | | Standard | error of |
| Variables | Mean | deviation | the mean | Mean | deviation | the mean |
| MW | 236.37 | 156.66 | 6.90 | 417.55 | 424.61 | 7.91 |
| #Heavy atoms | 16.77 | 11.09 | 0.49 | 28.44 | 28.79 | 0.54 |
| #Aromatic heavy | 5.87 | 6.07 | 0.27 | 8.97 | 9.37 | 0.17 |
| atoms | | | | | | |
| Fraction Csp3 | 0.44 | 0.35 | 0.02 | 0.46 | 0.25 | 0.00 |
| #Rotatable bonds | 3.18 | 5.09 | 0.22 | 7.44 | 13.05 | 0.24 |
| #H-bond acceptors | 3.73 | 3.03 | 0.13 | 6.13 | 8.98 | 0.17 |
| #H-bond donors | 2.02 | 1.95 | 0.09 | 2.71 | 5.45 | 0.10 |
| MR | 64.68 | 42.64 | 1.88 | 109.77 | 105.10 | 1.96 |
| TPSA | 68.93 | 51.81 | 2.28 | 114.60 | 192.21 | 3.58 |
| iLOGP | 1.78 | 1.46 | 0.06 | 1.79 | 6.20 | 0.12 |
| XLOGP3 | 1.66 | 2.86 | 0.13 | 2.06 | 3.44 | 0.06 |
| WLOGP | 1.66 | 2.35 | 0.10 | 2.13 | 3.49 | 0.07 |
| MLOGP | 0.95 | 2.13 | 0.09 | 1.13 | 3.17 | 0.06 |
| Silicos-IT Log P | 1.80 | 2.32 | 0.10 | 2.46 | 3.15 | 0.06 |
| Consensus Log P | 1.57 | 2.09 | 0.09 | 1.91 | 3.02 | 0.06 |
| ESOL Log S | −2.39 | 2.24 | 0.10 | −3.52 | 2.70 | 0.05 |
| ESOL Solubility | 1054.94 | 9228.83 | 406.67 | 507237.11 | 25549898.10 | 476094.24 |
| (mg/ml) | | | | | | |
| ESOL Solubility | 4.66 | 29.97 | 1.32 | 697.65 | 35781.71 | 666.75 |
| (mol/l) | | | | | | |
| Ali Log S | −2.72 | 2.92 | 0.13 | −4.13 | 4.09 | 0.08 |
| Ali Solubility | 8310.29 | 147912.99 | 6517.83 | 1.92E+10 | 1.03E+12 | 1.92E+10 |
| (mg/ml) | | | | | | |
| Ali Solubility | 54.41 | 1006.54 | 44.35 | 2.70E+5 | 1.45E+7 | 2.69E+5 |
| (mol/l) | | | | | | |
| Silicos-IT LogSw | −2.57 | 2.55 | 0.11 | −4.53 | 3.48 | 0.06 |
| Silicos-IT Solubility | 13462.50 | 181670.56 | 8005.36 | 6.85E+13 | 3.40E+15 | 6.34E+13 |
| (mg/ml) | | | | | | |
| Silicos-IT Solubility | 38.70 | 442.96 | 19.52 | 4.36E+10 | 2.19E+12 | 4.07E+10 |
| (mol/l) | | | | | | |
| log Kp (cm/s) | −6.57 | 1.86 | 0.08 | −7.43 | 3.86 | 0.07 |
| Synthetic | 2.71 | 1.66 | 0.07 | 3.93 | 1.92 | 0.04 |
| Accessibility | | | | | | |

Secondly, the average similarity between compounds with the same medicinal effects and randomly selected drugs were compared. It was confirmed that the average similarity of compounds with the same medicinal effect was 0.259±0.031, whereas the average similarity of randomly selected compounds was 0.091±0.014. This result indicates that the chemical properties of compounds with the same medicinal effect are likely to be similar.

### Experimental Example 4: Learning of Deep Learning Model

4-1. Generalization of Output

Latent knowledge, molecular interaction, and chemical property features of natural compounds or drugs were used as input features of the deep learning model. To predict the potential effects list from the input features, 15 deep learning models for 15 diseases were constructed.

Hidden layers generalized the outputs by providing a high-level representation that was more abstract than the previous layer by discovering nonlinear relationships between the low- and high-level data. Xₗ is the output of the l-th hidden layer. The forward propagation of the neural network with l-th hidden layer can be represented by Equation 2.

Xₗ=f(WₗXₗ₋₁+bₗ) [Equation 2]

where Wₗ=[wₗ₁, wₗ₂, . . . , wₗₙ] is the weight matrix of the edge from (l−1)-th layer to l-th layer, bₗ is the bias of each hidden units, and f(·) is the activation function.

4-2. Application of Partially Connected Structure

The hidden layers are divided into two parts: the partially connected and fully connected parts. A fully connected neural network is the most commonly used model because it usually does not need a priori information on input data for defining the structure of the model. The fully connected neural network simplifies the model design since every neuron in one layer is connected to every neuron in the next layer. However, the fully connected neural network may need large training data, and cannot consider the characteristic of the input feature types.

A partially connected neural network may be defined as a network that contains only a subset of all possible connections. The partially connected neural network has strengths in reducing complexity and improving generalization without producing modeling errors. The deep learning model of the present disclosure applied a partially connected network to learn the spatially distinguished representation of each feature.

When input neurons are connected to the next layer of neurons, it was set that the input neurons were connected to only the same input feature type of neurons. In the above-mentioned weight matrix (Wₗ), zero values are set for the disconnected edges based on feature types.

When n input features are fully connected to m neurons included in the hidden layer, n·m edges are created, but the deep learning model of the present disclosure creates Σᵢnᵢ·mᵢ edges (where i is the number of feature types). That is, the partially connected model of the present disclosure generated 121,018 (=(101·68)+(285·190)+(300·200)) edges, whereas the fully-connected model generated 314,188 (=(101+285+300)·(68+190+200)) edges.

A partially connected structure was applied to the first and second hidden layers. This process reduced the number of edges to be trained by about 37%. Therefore, the weights of the edges could be learned with a relatively small training set taking into account the input feature types. The outputs of each partially connected layer are further concatenated to produce the single layer.

4-3. Batch Normalization

The rectified linear unit (ReLU) activation function of f(x)=max (0, x) was applied to all hidden units to increase the nonlinearity. The weights were initialized using random numbers with zero-centered Gaussian with standard deviation of √(2/nₗ) (nₗ is the number of input units) that takes into account the ReLU nonlinearity.

The batch normalization was performed to normalize the input layer by re-centering and re-scaling. The class-weighted binary cross-entropy loss function for gradient descent was used to handle imbalanced dataset and defined by Equation 3 below.

$${L}_{w} = - {\sum }_{i} {w}_{0} {y}_{i} log ( {\hat{y}}_{i} ) + {w}_{1} ( 1 - {y}_{i} ) log ( 1 - {\hat{y}}_{i} ) \qquad [ Equation 3 ]$$

where i is the number of samples, ŷᵢ is the predicted model output, and yᵢ is the corresponding target value. w₀ and w₁ are the weights for class 1 and 0, which are set to be inversely proportional to the class frequencies. To optimize the loss function, the Adam optimizer was applied with the learning rate=0.0001, the learning rate decay=0, β₁=0.9, and β₂=0.999.

4-4. Learning of Input Features

When the input features are complex and heterogeneous, the deep learning model can improve the prediction performance by learning high-level representation from low-level features.

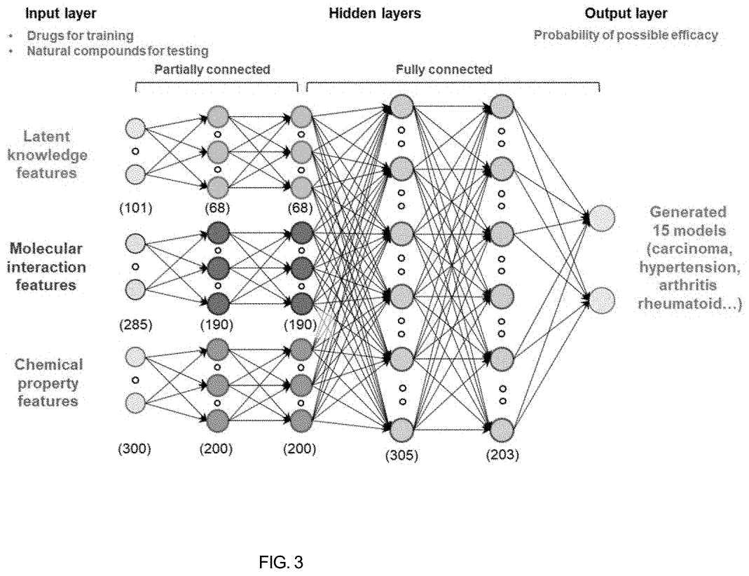

The deep learning model of the present disclosure consists of four sequential layers: (i) an input layer, (ii) partially connected hidden layers, (iii) fully connected hidden layers, and (iv) an output layer (FIG. 3).

To avoid overfitting, early stopping was applied to an iterative procedure of gradient descent, and the model for 3,000 epochs and a batch size of 64 were run (patience=30).

A total of 2,882 approved and investigational drugs were used to train the model, and 4,507 natural compounds were used for testing. To train the model, the output layer needed data indicating the medicinal effects of the drugs.

The medicinal effect information in DrugBank is described using free text, named entity recognition (NER), and thus was applied to extract disease terms with standard identifiers. The disease terms were extracted from the medicinal effect information by using a bidirectional encoder representation from transformers (BERT)-based NER tool (referred to as BERN).

The extracted disease terms were mapped to medical subject headings (MeSH) IDs and then converted into class labels. For each drug, an average of 2.57±0.11 (confidence interval=0.95) MeSH IDs were mapped. In the deep learning model of the present disclosure, out of a total of 1,607 diseases, 15 disease terms that most frequently appeared in the medicinal effect information of drugs were used for predictions of disease terms.

### Experimental Example 5: Medicinal Effect Prediction and Performance Evaluation of Natural Compounds

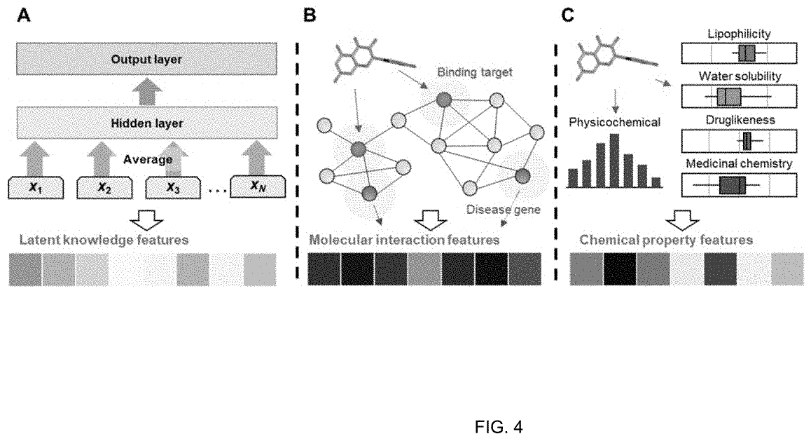

The medicinal effects of natural compounds were predicted by the deep learning model constructed using three types of feature data (FIG. 4). For all natural compounds and drugs, the algorithm works in four steps: (i) collecting various types of natural compound and drug information from public databases; (ii) generating latent knowledge, molecular interaction, and chemical property features from the collected information via text mining, network analysis, and chemical property analysis; and (iii) training the deep learning model by using the features of the approved and investigational drugs as inputs and the verified medicinal effect information as outputs; and (iv) predicting the medicinal effects of natural compounds based on the trained deep learning model.

5-1. Analysis of Area Under the ROC Curve (AUROC)

To assess the performance of the deep learning model, the AUROC was calculated. The performance for two different types of model structures and four different types of input data was tested through the following five combinations: (i) a partially connected model using all features; (ii) a fully connected model using all features; (iii) a fully connected model using the latent knowledge feature only; (iv) a fully connected model using the molecular interaction feature only; and (iv) a fully connected model using the chemical property feature only.

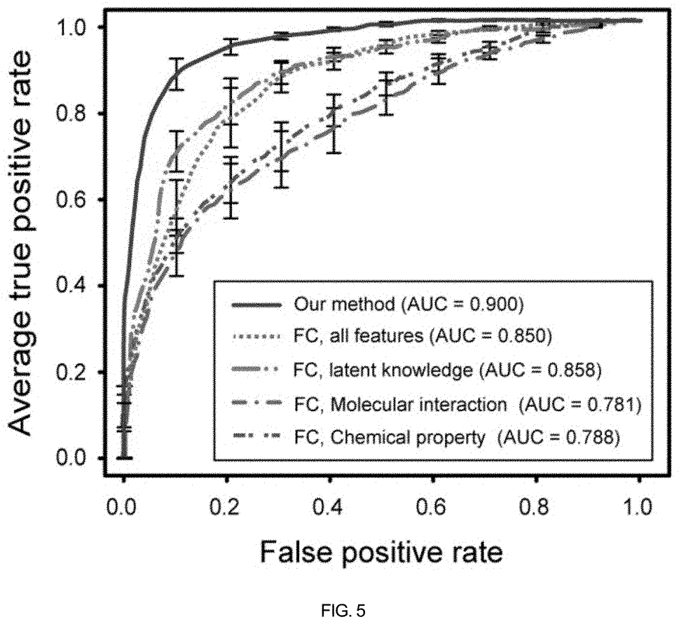

The 10-fold cross-validation was performed using only drug information. The drugs were divided at a ratio of 6:2:2 to train, validate, and test the model. AUROC values for 15 diseases were obtained, and shown in FIG. 5 and Table 2.

TABLE 2

| | Partially | | | | |
|---|---|---|---|---|---|
| | connected | Fully connected | | | |
| | All features | | Latent | Molecular | Chemical |
| | (exemplary | | knowledge | interaction | property |
| Disease term | embodiment) | All features | features only | features only | features only |
| Carcinoma | 0.774 | 0.684 | 0.767 | 0.702 | 0.711 |
| Hypertension | 0.970 | 0.962 | 0.955 | 0.882 | 0.777 |
| Pain | 0.943 | 0.776 | 0.840 | 0.815 | 0.611 |
| Diabetes mellitus, | 0.850 | 0.765 | 0.824 | 0.564 | 0.616 |
| type 2 | | | | | |
| Arthritis, rheumatoid | 0.774 | 0.692 | 0.692 | 0.683 | 0.667 |
| Urinary tract | 0.985 | 0.983 | 0.948 | 0.986 | 0.944 |
| infections | | | | | |
| Alzheimer's | 0.864 | 0.757 | 0.859 | 0.588 | 0.810 |
| disease | | | | | |
| Bacterial infections | 0.948 | 0.926 | 0.880 | 0.717 | 0.865 |
| Parkinson's | 0.995 | 0.947 | 0.977 | 0.913 | 0.953 |
| disease | | | | | |
| Heart failure | 0.880 | 0.873 | 0.865 | 0.727 | 0.833 |
| Sleep initiation and | 0.875 | 0.846 | 0.865 | 0.669 | 0.870 |
| maintenance | | | | | |
| disorders | | | | | |
| Skin diseases | 0.774 | 0.789 | 0.759 | 0.587 | 0.653 |
| Nausea | 0.934 | 0.971 | 0.865 | 0.957 | 0.798 |
| Myocardial | 0.964 | 0.798 | 0.800 | 0.975 | 0.766 |
| infarction | | | | | |
| Stroke | 0.972 | 0.974 | 0.971 | 0.946 | 0.949 |
| Average | 0.900 ± 0.040 | 0.850 ± 0.054 | 0.858 ± 0.042 | 0.781 ± 0.077 | 0.788 ± 0.059 |

As can be confirmed in FIG. 5 and Table 2, the partially connected model using all features (avg. AUROC=0.900±0.040) exhibited better performance than the model using only single information (avg. AUROC=0.781±0.077 to 0.858±0.042).

The fully connected model using all features (avg. AUROC=0.850±0.054) exhibited worse performance than the fully connected model using the latent knowledge feature only. This is because the number of training samples is insufficient compared to the number of weights to be learned in fully connected model using all features. The partially connected model could be trained by a relatively smaller data set compared with the fully connected model, and thus exhibited better performance compared with the full connected model.

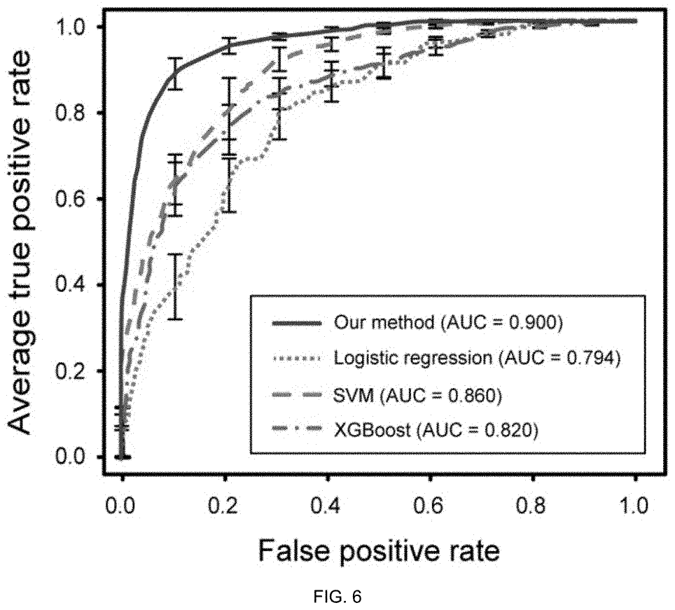

Next, the exemplary embodiment of the present disclosure was compared with other machine learning methods, such as logistic regression, support vector machine (SVM), and bootstrapping, and the results are shown in FIG. 6 and Table 3.

TABLE 3

| | Exemplary | Logistic | | |
|---|---|---|---|---|
| Disease term | embodiment | regression | SVM | XGBoost |
| Carcinoma | 0.774 | 0.673 | 0.715 | 0.752 |
| Hypertension | 0.970 | 0.827 | 0.846 | 0.878 |
| Pain | 0.943 | 0.761 | 0.793 | 0.822 |
| Diabetes mellitus, type 2 | 0.850 | 0.714 | 0.766 | 0.810 |
| Arthritis, rheumatoid | 0.774 | 0.653 | 0.688 | 0.725 |
| Urinary tract infections | 0.985 | 0.903 | 0.934 | 0.952 |
| Alzheimer's disease | 0.864 | 0.772 | 0.817 | 0.831 |
| Bacterial infections | 0.948 | 0.851 | 0.826 | 0.916 |
| Parkinson's disease | 0.995 | 0.910 | 0.952 | 0.963 |
| Heart failure | 0.880 | 0.813 | 0.807 | 0.833 |
| Sleep initiation and | 0.875 | 0.751 | 0.796 | 0.855 |
| maintenance disorders | | | | |
| Skin diseases | 0.774 | 0.725 | 0.740 | 0.781 |
| Nausea | 0.934 | 0.812 | 0.912 | 0.892 |
| Myocardial infarction | 0.964 | 0.836 | 0.881 | 0.893 |
| Stroke | 0.972 | 0.915 | 0.964 | 0.967 |
| Average | 0.900 ± 0.040 | 0.794 ± 0.042 | 0.829 ± 0.043 | 0.858 ± 0.038 |

As can be confirmed in FIG. 6 and Table 3, the deep learning model of the exemplary embodiment (avg. AUROC=0.900±0.040) exhibited better performance than other machine learning methods (avg. AUROC=0.781±0.077 to 0.858±0.042).

Moreover, the average prediction accuracy of the model for 15 diseases of the present disclosure was measured to be 0.971±0.011.

These results indicate that the deep learning model of the exemplary embodiment is well built to exhibit a high accuracy of medicinal effect prediction of natural compounds by reflecting the characteristics of the heterogeneous information.

5-2. Prediction of Medicinal Effects of Natural Compounds

To predict the medicinal effects of natural compounds, the deep learning model was trained based on drug information and the accuracy of medicinal effect prediction of the model was investigated through the verified effect information of the natural compounds.

An additional experiment was conducted using the inferred effects of the natural compounds as a test set because the verified medicinal effect information of natural compounds was limited. The results are shown in Table 4.

TABLE 4

| Disease term | Verified effect | Verified and inferred effect |
|---|---|---|
| Carcinoma | 0.767 | 0.813 |
| Hypertension | 0.912 | 0.935 |
| Pain | 0.871 | 0.903 |
| Diabetes mellitus, type 2 | 0.793 | 0.822 |
| Arthritis, rheumatoid | 0.725 | 0.761 |
| Urinary tract infections | 0.846 | 0.910 |
| Alzheimer's disease | 0.827 | 0.841 |
| Bacterial infections | 0.879 | 0.927 |
| Parkinson's disease | 0.924 | 0.961 |
| Heart failure | 0.808 | 0.894 |
| Sleep initiation and | 0.797 | 0.867 |
| maintenance disorders | | |
| Skin diseases | 0.718 | 0.785 |
| Nausea | 0.844 | 0.913 |
| Myocardial infarction | 0.902 | 0.947 |
| Stroke | 0.870 | 0.969 |
| Average | 0.832 ± 0.032 | 0.883 ± 0.033 |

As can be confirmed in Table 4, the deep learning model, which was trained using drug information, successfully predicted the verification effects (avg. AUROC=0.832±0.032) and verification and inference effects (avg. AUROC=0.883±0.033) of natural compounds.

5-3. Calculation of List of Disease Scores for Drugs and Statistical Analysis

The statistical analysis was performed based on literature reporting the predicted medicinal effects of natural compounds. The sum of protein values of the molecular interaction features based on the therapeutic target and biomarker information of diseases was mapped to 3,832 diseases defined by MeSH and the on-line Mendelian inheritance in man (OMIM), and then a list of disease scores for 67,605 drugs was obtained.

Three independent sets were made by selecting a top-ranked 10% set, a bottom-ranked 10% set, and a randomly selected set (random set). It was investigated whether the high-scored predictions have more evidence than the low-scored and randomly selected predictions.

To do this, the co-occurrences (n_{c}) of natural compounds and disease terms in PubMed abstracts were counted. The average co-occurrence frequency of the high-scored set was calculated to be 0.87±0.18, which was 9.6 and 3.8 times larger than the low-scored set (0.09±0.03) and the random set (0.23±0.11).

Thereafter, the co-occurrence was normalized as the Jaccard index (JI) by dividing the frequency of co-occurrence by the frequency of the union of individual terms to reduce the size influence associated with the term frequency. The average Jaccard index of the high-scored set was 1.07×10⁻⁴, which was higher than those of the low-scored set (2.17×10⁻⁸) and the random set (4.31×10⁻⁵).

Furthermore, Fisher's exact test was performed to examine the significance of the predictions. Fisher's exact test assesses the null hypothesis, for example, “there is no difference in the proportions of predictions between natural compound and disease”, of independence based on the hypergeometric distribution of the numbers in a contingency table.

To obtain the contingency table of each prediction, the number of PubMed abstracts was counted based on whether they included the natural compound and whether they included the target disease. The number of significant predictions of the high-scored set (n_{f}=58.53±14.01) was markedly larger than those of the low-scored set (n_{f}=13.46±7.42) and random set (n_{f}=27.86±9.98).

Lastly, the Mann-Whitney U test was performed to confirm the statistically significant difference among the high-scored, low-scored, and random sets was significant, and the results are synthetically shown in Table 5.

TABLE 5

| | | Co- | Jaccard | Fisher's exact |
|---|---|---|---|---|
| | | occurrence | index | test |
| High-scored set, H | | 0.87 ± 0.18 | 1.07 × 10⁻⁴ | 58.53 ± 14.01 |
| Low-scored set, L | | 0.09 ± 0.03 | 2.17 × 10⁻⁸ | 13.46 ± 7.42 |
| Random set, R | | 0.23 ± 0.11 | 4.31 × 10⁻⁵ | 27.86 ± 9.98 |
| Mann-Whitney U | H vs L | <0.001 | <0.001 | <0.001 |
| test (p-value) | H vs R | <0.001 | <0.001 | <0.001 |
| | L vs R | <0.001 | <0.001 | <0.001 |

A p-value of Mann-Whitney U test lower than 0.05 was considered statistically significant. Referring to FIG. 5, all the p-values were calculated to be lower than 0.001, indicating that the analysis results were significantly different among the high-scored, low-scored, and random sets.

5-4. Evidence-Based Analysis

5-4-1. In Vitro and Animal Studies

5-Caffeoylquinic acid (COA) may prevent cognitive impairment in mice with Alzheimer's disease. Tangeretin may have therapeutic effects on rheumatoid arthritis in a rat model. Gossypol family members, such as BH3 mimetics, may have benefits in the management of rheumatoid arthritis. Indolyl-methyl-glucosinolate was reported to exert anti-inflammatory activity, and gentianine showed low anti-inflammatory activity in carrageenan-induced hind-paw edema. Gambogic acid may ameliorate angiogenesis in mice with diabetic retinopathy. Gamma-oryzanol was shown to be safe and effective in improving the conditions of diabetes mellitus in several animal studies. Octopamine may be involved in central blood pressure regulation. According to the reperfusion duration, route of administration, and timing of the pretreatment regimen, resveratrol showed benefits in the conservative treatment of myocardial infarct-sparing. N-methyl-(R) salsolinol, as an endogenous neurotoxin, may induce Parkinson's disease in rats. The proliferation of MDA-MB-231 cells is prohibited using neohesperidin in a time- and dose-dependent manner in human breast adenocarcinoma. Tritiated norephedrine may inhibit the substitution of betaphenylethylamines in rats. Agmatine protected brain tissues from edema after cerebral ischemic.

The results of the in vitro and animal studies were collected as evidence.

5-4-2. Clinical Studies

Melatonin may enhance the therapeutic effects of various anticancer drugs. Ergosterol biosynthesis inhibitors may exhibit curative activities in murine models of acute and chronic Chagas disease. In patients with chronic congestive heart failure, L-arginine prolongs the exercise duration. Reserpine may reduce the systolic blood pressure as a first-line antihypertensive drug. Plasma norepinephrine is directly related to muscle sympathetic nerve activity values in hypertensive group. In a blind placebo-controlled trial, a pyridoxine-doxylamine combination appears to be safe for pregnant women suffering from nausea and vomiting associated with pregnancy. The randomized controlled trials (RCTs) showed that Zingiber officinale Roscoe, which contains camphene, can be typically used to alleviate nausea and vomiting in pregnant women. In a randomized double-blind crossover study, the use of oral morphine for pain control led to a reduction in pain intensity relative to placebo use. Eugenol and carvacrol were shown to induce oral irritation, causing various types of pain. A single patch containing methyl salicylate and l-menthol significantly relieved the pain associated with mild to moderate muscle strain. Laudanosine is a neurotoxin that promotes Parkinson's disease, and prevents NADH-linked mitochondrial respiration and complex I activity. Melatonin decreases sleep onset latency, increases total sleep time, and improves overall sleep quality, as shown in the meta-analysis. One case study revealed that long-term colchicine therapy leads to symptomatic respiratory muscle weakness. Clopidogrel monotherapy leads to lower risks of major adverse cardiovascular or cerebrovascular events compared with aspirin treatment. The demethylation of 5-methylcytosine may help in the management of interstitial cystitis. Flucytosine may serve as an effective and safe treatment for urinary tract infection.

These clinical study results were collected as evidence, and are summarized in Table 6 together with the results of in vitro and animal studies.

TABLE 6

| | | Animal and clinical |
|---|---|---|
| | | studies (PubMed |
| Disease term | Compound term | identifier) |
| Alzheimer's | 4,5-dicaffeoylquinic acid | PMID: 32075202 |
| disease | 3,4-dicaffeoylquinic acid | PMID: 32075202 |
| Rheumatoid | Tangeretin | PMID: 31344704 |
| arthritis | Gossypol | PMID: 23974697 |
| Bacterial infection | Indolylmethylglucosinolate | PMID: 24360830 |
| | Gentianamine | PMID: 12805773 |
| Carcinoma | Melatonin | PMID: 28415828 |
| Diabetes mellitus, | Gambogic acid | PMID: 29129773 |
| type 2 | Gamma-oryzanol | PMID: 26718022 |
| Heart failure | Ergosterol | PMID: 19753490 |
| | Arginine | PMID: 15226784 |
| Hypertension | Reserpine | PMID: 27997978 |
| | Norepinephrine | PMID: 29915014 |
| | Octopamine | PMID: 6125331 |
| | Digitoxin | PMID: 26321114 |
| Myocardial | Resveratrol | PMID: 31182995 |
| infarction | | |
| Nausea | Pyridoxine | PMID: 25884778 |
| | Camphene | PMID: 29614764 |
| Pain | Morphine | PMID: 8544547 |
| | Carvacrol | PMID: 23791894 |
| | L-menthol | PMID: 20171409 |
| Parkinson's | Salsolinol | PMID: 9120428 |
| disease | dl-Laudanosine | PMID: 8769881 |
| Skin disease | Neohesperidin | PMID: 23285810 |
| Sleep initiation | Norephedrine | PMID: 26321114 |
| and maintenance | Melatonin | PMID: 23691095 |
| disorders | Colchine | PMID: 14744269 |
| Stroke | Aspirin | PMID: 31867054 |
| | Agmatine | PMID: 20029450 |
| Urinary tract | 5-Methylcytosine | PMID: 7767983 |
| infection | Cytosine | PMID: 2041144 |

Therefore, the use of the present disclosure mitigates the bottleneck effect of the deep learning model by utilizing large amounts of heterogeneous information containing latent knowledge, molecular interactions, and chemical properties, to mitigate the incomplete information, and thus, the present disclosure can be used to perform a large-scale natural compound study. Furthermore, this approach can be used in a preliminary screening of compounds for a large number of candidate medicinal substances.

What is claimed is:

1. A method for predicting medicinal effects of compounds by using deep learning, the method comprising:
a data acquirement step of acquiring medicinal substance data;
a feature generation step of generating feature data from the acquired medicinal substance data;
a training step of training a neural network model including an input layer, hidden layers, and an output layer as feature data; and
a prediction step of predicting medicinal effects of compounds by applying compound data to the neural network model.

2. The method of claim 1, wherein the feature data has a fixed-length numeric vector form.

3. The method of claim 1, wherein the feature data includes latent knowledge features, molecular interaction features, and chemical property features.

4. The method of claim 3, wherein the latent knowledge features are generated through word embedding.

5. The method of claim 4, wherein the word embedding is performed using at least one selected from the group consisting of Word2vec, AdaGram, fastText, and Doc2vec.

6. The method of claim 3, wherein the molecular interaction features are generated by constructing a protein-protein interaction (PPI) network from the acquired compound data and medicinal substance data and applying a random walk with restart (RWR) algorithm thereto.

7. The method of claim 3, wherein the chemical property features are generated through SwissADME.

8. The method of claim 1, wherein:
the hidden layers include partially connected layers and fully connected layers; and
the input layer, partially connected layers, fully connected layers, and output layer are arranged in that order in the neural network model.

9. The method of claim 1, wherein the hidden layers include rectified linear unit (ReLU) and batch normalization functions.

10. The method of claim 1, wherein the compound data are acquired from at least one type of database selected from the group consisting of Korean Traditional Knowledge Portal (KTKP), Traditional Chinese Medicine Integrated Database (TCMID), Compound Combination-Oriented Natural Product Database with Unified Terminology (COCONUT), and Food Database (FooDB).
